(19) United States
(12) Patent Application Publication
(10) Pub. No.: US 2025/0161171 A1
(43) Pub. Date: May 22, 2025

(54) TOOTHPASTE TABLET HAVING XYLITOL AND NANO-HYDROXYAPATITE AND ASSOCIATED METHOD OF MAKING

(71) Applicant: BIOM, LLC, Sheridan, WY (US)

(72) Inventor: Ilon Choai, Sheridan, WY (US)

(21) Appl. No.: 18/932,675

(22) Filed: Oct. 31, 2024

Related U.S. Application Data
(60) Provisional application No. 63/601,770, filed on Nov. 22, 2023.

(51) Int. Cl. A61K 8/02; A61K 8/19; A61K 8/24; A61K 8/25; A61K 8/34; A61K 8/73; A61Q 11/00

(52) U.S. Cl. CPC A61K 8/022; A61K 8/19; A61K 8/24; A61K 8/25; A61K 8/345; A61K 8/731; A61Q 11/00

(57) ABSTRACT

A toothpaste composition tablet may include xylitol and nano-hydroxyapatite in a concentration greater than about 4.75% by mass (w/w) of the composition, and preferably between about 4.75% and about 5.25% by mass (w/w). The xylitol in an example may be between about 48.5% and about 53.5% by mass (w/w) of the composition and mixed with a mint flavoring agent, cellulose, calcium carbonate, and an anionic surfactant. The xylitol may be between about 14.25% and about 15.75% by mass (w/w) of the composition and may include sorbitol and glycerin.

### PRIORITY APPLICATION(S)

The application is based upon U.S. provisional patent Application No. 63/601,770 filed Nov. 22, 2023, the disclosure which is hereby incorporated by reference in its entirety.

### FIELD OF THE INVENTION

The present invention relates to the field of oral health, and more particularly, this invention relates to a toothpaste composition formulated into toothpaste tablets and related methods of making the toothpaste tablet.

### BACKGROUND OF THE INVENTION

Cavities, also sometimes referred to as caries, are parenchymal defects caused by decalcification of the tooth by organic acids that are produced by the oral bacteria present on the tooth surface. It has been determined in most cases that subsurface demineralization occurs before a cavity appears. The initial decay state may indicate that some tooth loss has occurred. The surface of the tooth is retained, but calcium has been lost under the surface. In some cases, there is a white layer with a high degree of calcification in the outermost layer of the enamel and a slight black layer under it, which in many cases corresponds to the demineralized portion of the tooth. When the initial caries occurs, the loss of calcium causes the crystal structure to change, and the tooth surface appears white. Because the caries or cavity is a real defect, it cannot be repaired naturally. The defective portion of the tooth cannot be filled without treatment by a dentist. In some cases, dental demineralization can be restored naturally with remineralization in the oral cavity.

For this initial decay state of the cavity, fluoride is often used to treat teeth because it strengthens the tooth structure. However, when fluoride is applied at high concentrations, only the surface layer is strongly remineralized, while the demineralized layer below the surface is not recalcified. The tooth surface remains as a white spot and is solid, but the phosphate ions, calcium, and other minerals do not reach the layer below it so there is not a natural remineralization.

It is known that nano-hydroxyapatite containing pastes may aid remineralization of teeth that have been damaged by the initial demineralized segments. The nano-hydroxyapatite replaces the minerals the teeth lost and fills in the tiny fissures in the teeth that bacteria would otherwise attack. It is also biocompatible with teeth since it is formed of similar material with the tooth composition.

The remineralization of the tooth enamel permits the reintroduction of mineral ions into the demineralized enamel. Hydroxyapatite has been found to be a primary component of enamel in the teeth. During demineralization, the calcium and phosphorus ions are drawn out from the hydroxyapatite. The mineral ions are introduced during remineralization to restore the structure of the hydroxyapatite crystals. When fluoride ions are present during the remineralization, through water fluoridation or the use of fluoride-containing toothpaste, stronger and more acid-resistant fluorapatite crystals are formed instead of the hydroxyapatite crystals. This hinders the remineralization of the teeth. Therefore, fluoride is not desired in many toothpastes or tablets that employ nano-hydroxyapatite and other hydroxyapatite components since the fluoride may effectively counter the positive effects of the nano-hydroxyapatite.

Recently many users have switched to chewable toothpaste tablets because they are environmentally friendly, compact, solid, and easy to pack and travel since they can also pass through security. They are beneficial for those that want to clean their teeth after every meal. The tablets may be packaged in recyclable or biodegradable materials, such as glass, tin or compostable pouches, helping reduce plastic waste. Each tablet usually includes a precise amount of ingredients, giving much greater control over the positive benefits of having a range of specific ingredients that may work together. Since tablets often do not contain fluoride, the more precise range of other ingredients, such as nano-hydroxyapatite, becomes important, which is also important when natural components and ingredients are used.

Also, special care must be made if a toothpaste composition is formulated for use as a children's toothpaste and especially a toothpaste tablet since children have delicate oral care needs. Many toothpaste tablets are known to include nano-hydroxyapatite, but its incorporation with other ingredients can make presenting a balanced toothpaste tablet challenging, especially a toothpaste tablet for children. There is a need to make a toothpaste tablet, and especially one for children having nano-hydroxyapatite for tooth remineralization not only to protect the gums and oral cavity, but also to provide a pleasant taste, destroy bacteria, and be palpable for human and more particularly, children's use in a tablet form.

### SUMMARY OF THE INVENTION

A toothpaste composition is formulated as a toothpaste tablet and may comprise xylitol in a concentration between about 48.5% and about 53.5% by mass (w/w) of the composition, and a mint flavoring agent in a concentration between about 11.4% and about 12.6% by mass (w/w) of the composition. Cellulose may be in a concentration between about 11.2% and about 12.3% by mass (w/w) of the composition and calcium carbonate may be in a concentration between about 6.2% and about 6.8% by mass (w/w) of the composition. An anionic surfactant may be in a concentration between about 5.0% and about 5.6% by mass (w/w) of the composition, and nano-hydroxyapatite may be in a concentration between about 4.75% and about 5.25% by mass (w/w) of the composition. The toothpaste composition is formulated into the tablet form.

Xanthan gum may be in a concentration between about 1.9% and about 2.1% by mass (w/w) of the composition. Sodium Cocoyl Isethionate (SCI) may be in a concentration between about 1.8% and about 2.0% by mass (w/w) of the composition. Ammonium glycyrrhizinate may be in a concentration between about 1.8% and about 2.0%. by mass (w/w) of the composition, and silica dioxide may be in a concentration between about 1.05% and about 1.2% by mass (w/w) of the composition. At least one of the following may be included, e.g., menthol, zinc citrate and sodium bicarbonate.

In another example, a toothpaste composition that is formulated into a toothpaste tablet may comprise glycerin in a concentration between about 22.5% and about 25.5% by mass (w/w) of the composition, and xylitol in a concentration between about 14.25% and about 15.75% by mass (w/w) of the composition. Sorbitol may be in a concentration between about 20.5% and about 23.5% by mass (w/w) of the composition, and nano-hydroxyapatite may be in a concentration between about 4.75% to about 5.25% by mass (w/w) of the composition. The toothpaste composition is formulated into the tablet form.

Silicon dioxide may be in a concentration between about 9.0% and about 11.0% by mass (w/w) of the composition, and fused silica may be in a concentration between about 1.9% and about 2.1% by mass (w/w) of the composition as an added mixed ingredient to the composition. Xanthan gum may be in a concentration between about 0.38% and about 0.42% by mass (w/w) of the composition and cellulose gum in a concentration between about 3.8% and about 4.2% by mass (w/w) of the composition as an added mixed ingredient to the composition. A sweetening agent extract may be in a concentration between about 0.14% and about 0.16% by mass (w/w) of the composition. A flavoring agent may be in a concentration between about 2.85% and about 3.15% by mass (w/w) of the composition. The xylitol may comprise between about 30 and about 60 mesh particle size. Deionized water may be included.

In another aspect, a method of forming a toothpaste composition as a toothpaste tablet may comprise mixing within a mixing vessel deionized water, xylitol in a concentration between about 14.25% and about 15.75% by mass (w/w), nano-hydroxyapatite in a concentration between about 4.75% to about 5.25% by mass (w/w), and a sweetening agent extract in a concentration between about 0.14% and about 0.16% by mass (w/w) until the sweetening agent extract is dissolved. This is followed by adding glycerin in a concentration between about 22.5% and about 25.5% by mass (w/w) to the mixing vessel and mixing until homogenous, which is followed by adding sorbitol in a concentration between about 20.5% and about 23.5% by mass (w/w) to the mixing vessel and mixing until homogenous.

This is followed by combining glycerin, xanthan gum in a concentration between about 0.38% and about 0.42% by mass (w/w) and cellulose gum in a concentration between about 3.8% and about 4.2% by mass (w/w) and stirring until smooth to form a slurry and adding the slurry to the mixing vessel and mixing until the xanthan gum and cellulose gum are fully hydrated. This is followed by adding silicon dioxide in a concentration between about 9.0% and about 11.0% by mass (w/w) and fused silica in a concentration between about 1.9% and about 2.1% by mass (w/w) to the mixing vessel and mixing until homogenous and smooth to form a toothpaste composition, which is then tableted into a toothpaste tablet, such as 600 mg.

The xylitol may comprise between about 30 and about 60 mesh particle size. A flavoring agent may be in a concentration between about 2.85% and about 3.15% by mass (w/w) and is added to the mixing vessel and mixed until homogenous before adding the silicon dioxide and fused silica. The sweetening agent extract may comprise Rebaudioside A. The method further comprises drying the toothpaste composition to a dry powder and tableting the dry powder to form the toothpaste composition tablet.

### BRIEF DESCRIPTION OF THE DRAWINGS

Other objects, features and advantages of the present invention will become apparent from the Detailed Description of the invention which follows, when considered in light of the accompanying drawings in which:

FIG. 1 is a table of a specific example of the component concentrations for the toothpaste composition as a children's toothpaste tablet.

FIG. 2 is a high-level flowchart of a method of forming the toothpaste composition as a toothpaste tablet having the composition components shown in the table of FIG. 1.

### DETAILED DESCRIPTION

Different embodiments will now be described more fully hereinafter with reference to the accompanying drawings, in which preferred embodiments are shown. Many different forms can be set forth and described embodiments should not be construed as limited to the embodiments set forth herein. Rather, these embodiments are provided so that this disclosure will be thorough and complete, and will fully convey the scope to those skilled in the art.

The toothpaste composition as described in detail below is formulated into toothpaste tablets that are a dentifrice providing enamel remineralization via a fluoride-free formulation. The toothpaste tablets are chewed and broken up into granules and pushed into gaps between the teeth and beneath the gums where brushes and paste cannot reach before reacting with saliva and foaming to clean teeth. The toothpaste composition includes several active ingredients, for example, nano-hydroxyapatite, that may be about 5% by mass (w/w) of the composition, and in an example, greater than 4.75% by mass (w/w) of the composition concentration formed from rod-shaped nano-hydroxyapatite particles.

The nano-hydroxyapatite ingredient operates in conjunction with other components for effective remineralization and long-term enamel protection. By incorporating nano-hydroxyapatite rather than fluoride, in addition to the other natural ingredients as explained below, the toothpaste tablets in this example are safe to swallow since nano-hydroxyapatite is known to be a safe, biocompatible material and the selection and range of other ingredients are selected to provide a balanced taste, excellent texture, and efficacy in the tablet, as well as operate as an antibacterial.

This tablet composition allows a convenient and environmentally friendly tablet that some dental experts consider better than the more common toothpaste since it permits more effective cleaning and may be chewed to break into granules, while helping loosen surface debris from the teeth. As the toothpaste tablet is chewed, the granules are pushed into the gaps between the teeth and between the gums where the brushes and toothpaste often cannot reach before reacting with the saliva and fizzing into a cleaning foam that may be brushed. This provides a deep and safer teeth cleaning and fresher breath than the more common toothpaste. Also, toothpaste tablets are environmentally friendly since there is no longer a toothpaste tube that must be discarded, which often winds up in a landfill every year.

Tablets, on the other hand, may be stored in compact tubes or tins, which can be safely packed in luggage or handbags without spilling. Also, toothpaste tablets are more hygienic than toothpaste that is packaged as a squeezable paste in a tube. The toothpaste tablets may be free from harmful preservatives that are often loaded into semi-liquid toothpaste, e.g., chemical preservatives and additives that prevent foaming of the toothpaste in the mouth rather than in the tube. Example chemical preservatives often found in commercially available toothpaste compositions include surfactants, such as SLS (sodium lauryl sulfate), which may irritate soft tissues in the mouth and cause ulcerations, and thus, especially unacceptable in a children's toothpaste composition as a tablet.

The composition in an example includes a blend of ingredients for the toothpaste tablets, including but not limited to xylitol, cellulose, a flavoring agent, nano-hydroxyapatite, zinc citrate, ammonium glycyrrhizinate (licorice root), sodium bicarbonate, calcium carbonate, Sodium Cocoyl Isethionate (SCI), and silica dioxide, which is formulated to tackle plaque. This blend has a dual-action mechanism and helps reduce existing plaque accumulation and prevent further buildup. Additionally, the composition, in this example as a tablet, aids in mitigating the sulfur compounds typically found in bad breath, thereby promoting fresher breath. This innovative composition is driven, in an example, by a holistic approach towards maintaining oral hygiene by addressing multiple concerns via one product.

The toothpaste is formulated as toothpaste tablets and includes a high xylitol content, which in this example, is greater than 48.5% by mass (w/w) of the composition and strategically formulated to stimulate saliva production, operating as a natural defense against bacteria. Most commercially available toothpastes have a lower xylitol content of about 5% to about 35% by volume, but the greater amount in the toothpaste composition as described helps remineralize in conjunction with the nano-hydroxyapatite and starve bad bacterial, but not in excess to disrupt oral and gut microbiomes.

Moreover, this larger amount of xylitol serves as a selective antibacterial agent and preserves the beneficial bacteria that are integral to a balanced oral microbiome while combating harmful bacteria without gastrointestinal discomfort. The toothpaste tablets are formulated with this broader understanding of oral ecology, promoting not just cleaner teeth, but a healthier oral environment.

The toothpaste tablets include the different components that are blended to facilitate compression into about 600 mg tablets, and in an example, about 550 to 650 mg tablets using a specialized tablet machine. This technique ensures a consistent dosage of active ingredients in each tablet, simplifying the user experience by eliminating the guesswork and waste associated with applying toothpaste using traditional toothpaste tubes. Moreover, the tablet form, accompanied by an eco-conscious air-tight glass jar packaging, underscores a user-friendly, waste-free, and environmentally responsible approach to dental hygiene.

A specific example of the different components and concentration is shown in the table below:

TABLE I

| | |
|---|---|
| 51.00% | Xylitol |
| 12.00% | Flavor such as mint |
| 11.70% | Cellulose |
| 6.50% | Calcium Carbonate |
| 5.30% | Anionic Surfactant |
| 5.00% | Nano-hydroxyapatite |
| 1.10% | Silica Dioxide |
| 2.00% | Xanthan Gum |
| 1.90% | Sodium Cocoyl Isethionate |
| 1.90% | Ammonium Glycyrrhizinate |
| 0.70% | Menthol |
| 0.50% | Zinc Citrate |
| 0.40% | Sodium Bicarbonate |

The example components are shown in the table above, but these components may vary in their concentration. For example, the xylitol may vary from about 48.5% to about 53.5% and be about 30 to 60 mesh size or 560 micrometers to as low as 250 micrometers during initial processing to form the tablet. The flavorant, such as the mint flavorant, may vary from about 11.4% to about 12.6%. The cellulose may vary from about 11.2% to about 12.3%. The xylitol and cellulose operate together during cross-linking to avoid a reaction leading to the formation of unsaturated compounds. The cellulose may also help thicken product during formulation.

The calcium carbonate may vary from about 6.2% to about 6.8%. An anionic surfactant and calcium carbonate are added together to improve the action of the calcium carbonate for constructive aspects of the nano-hydroxyapatite. An anionic surfactant may vary from about 5.0% to about 5.6%. It is possible to use Sodium Lauryl Sulfate (SLS) in an example, but other coconut derived surfactants may be used. Other surfactants that are not harsh on the gums and mouth may be used.

The nano-hydroxyapatite may vary from about 4.75% to about 5.25%, and in an example, may be about 20 to 100 nanometers, and preferred about 20 to 50 nanometers that match nanosized defects due to acid reaction at enamel. The silica dioxide may vary from about 1.05% to about 1.15%. The xanthan gum may vary from about 1.9% to about 2.1%. The Sodium Cocoyl Isethionate (SCI) may vary from about 1.8% to about 2.0% and is a surfactant derived from coconut oil and isethionic acid and helps foam and solidification. The sodium cocoyl isethionate (SCI) is a mild, high foaming anionic surfactant with high purity made from coconut fatty acids. As an example, it may have a minimum of 85% active matter with a free fatty acid of 14% maximum. It is compatible with soaps and anionic, non-ionic and amphoteric surfactants. SCI is a sodium salt of the coconut fatty acid ester of isethionic acid. The negative charge helps create a lather and lift contaminants from the teeth and oral cavity. Because it is manufactured using ethoxylation, it is used in a lower proportion with another anionic surfactant. For example, in may be in combination with the other anionic surfactant in a ratio of about 1:2.5 to 1:3.5. The example proportion is about 1:2.8. Although the other anionic surfactant may be sodium lauryl sulfate (SLS), others may be more preferred such as sodium dodecylbenzene sulfonate and ammonium lauryl sulfate. It may be possible to use cocamidopropyl betaine and sodium methyl cocoyl taurate (adinol) and other anionic surfactants.

The ammonium glycyrrhizinate may vary from about 1.8% to about 2.08. The methanol may vary from about 0.65% to about 0.75%, and the zinc citrate may vary from about 0.47% to about 0.53%. The sodium bicarbonate may vary from about 0.38% to about 0.42%. These components operate both as a singular agent and in combination with each other and some components provide synergy with each other.

The xylitol is a high percentage component of over 50% in an example and decreases the incidence of dental caries by increasing saliva flow and the pH and reduces the number of cariogenic and periodontopathic bacteria. The xylitol reduces plaque levels, xerostomia, gingival inflammation and erosion of teeth. Xylitol is a white crystalline solid that is freely soluble in water and is a polyalcohol and sugar alcohol, and more specifically, an alditol. It is one of three 5-carbonate sugar alcohols. The xylitol as a sugar alcohol contains a five carbon-polyol that is metabolized via the phosphor-enolpyruvate-phospho-transverse pathway that produces xylitol-5-phosphate as a product and competes with phosphofructokinase and inhibits glycolysis via the accumulation of glucose-6-phosphate.

The nano-hydroxyapatite not only aids in remineralization, but also helps reduce hypersensitivity that is thought to be regulated by fluid within dentinal tubules. This fluid movement may excite receptor cells in the pulp and trigger sensation of pain. The nano-hydroxyapatite may penetrate and seal these tubules and stop the circulation of the fluid and the sensation to pain. The small amount of zinc as zinc citrate, such as 0.5%, may form with the nano-hydroxyapatite a zinc-hydroxyapatite, such as zinc carbonate nanocrystal dentifrice that operates through a tubule plugging action.

Many bleaching agents and foods release reactive oxygen species that can degrade enamel. To prevent this, the nano-hydroxyapatite reduces the impact of these agents. The cellulose may also operate as a dressing type of material that adheres to the moist oral mucosa to form a temporary barrier protecting the gums and inside tissues of the oral cavity from irritation and pain.

The calcium carbonate may be used as a dental cleaning and polishing agent as it is soluble in water, but insoluble in alcohol. It has some antacid effect. The surfactants operate to decrease surface tension. The anionic surfactant had a head that may be negative. The silica dioxide or silicon dioxide may operate as a dental thickener and is cost-effective to build suitable levels of viscosity without compromising performance. The xanthan gum may operate as a stabilizer and provide uniformity and stability. It is a polysaccharide that may also operate as an emulsifier, stabilizer, and a thickening agent to prevent various components from separating. Even a small amount such as 1% produces a significant increase in the viscosity of a liquid. This is helpful when the tablet is chewed. The sodium cocoyl isethionate is naturally derived from coconut oil and operates as a mild foaming agent. Although in some cases of prior art formulations, the sodium cocoyl isethionate may cause dry mouth, the current formulation has been found not to have such issues of dry mouth.

The ammonium glycyrrhizate is an extract from the licorice root and adds a sweet taste to the tablets to mask the unpleasant flavors imparted by other ingredients. It is known to be 30 to 50 times as sweet as sucrose. Menthol is a natural chemical found in peppermint and other mint plants and can be produced synthetically. It reduces the harshness of other components and has a local anesthetic and counter-irritant quality to relieve any minor gum and throat irritations. It may operate as a weak K-opoid receptor agonist.

The zinc citrate may be available as a dihydrate and trihydrate and is produced from the neutralization of citric acid with a high purity zinc source and the subsequent precipitation and dehydration. It has antimicrobial and anti-inflammatory effects and reduces and inhibits the formation of dental plaque and tartar. The zinc citrate fights common colds and may be helpful. Sodium bicarbonate is used in a variety of oral care products.

An example methodology of forming the tablet is now described. The steps generally include:
1) Grinding of granular raw materials;
2) Drying of hygroscopic raw materials, such as the xylitol in powder form, the ammonium glycyrrhizinate and flavoring agent, such as mint, in ventilated ovens for 45 to 60 minutes at temperatures appropriate to each material within the range of 40° C. to 100° C.;
3) Sieving of raw materials according to the mesh size corresponding to each type of input based on particle size requirements;
4) Weighing of materials according to the assigned batch weight;
5) Mixing of post-dried hygroscopic agents with silicon dioxide for a predetermined processing time;
6) Addition of the remaining ingredients and mixing for a predetermined processing time;
7) Feeding the powder to a tableting machine previously adjusted and conditioned for the process;
8) Tableting process; and
9) Packaging of the tablet in its primary packaging and subsequently in its secondary packaging.

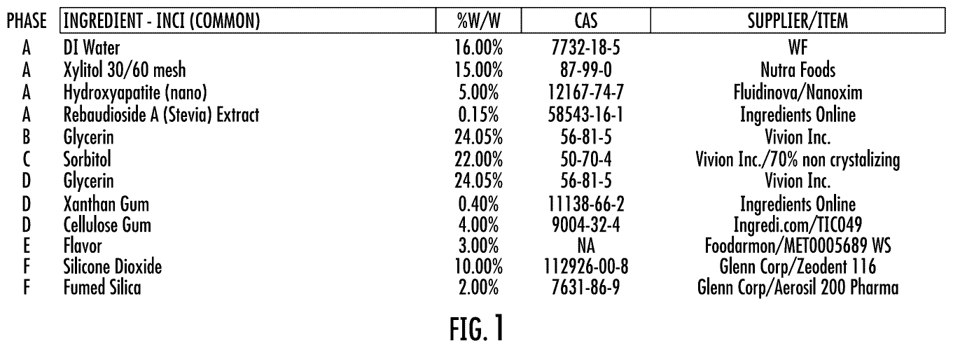

The tablet as described above is suitable for adult use, but young children under the age of 12 may require a less harsh tablet. The toothpaste composition may also be formulated into a toothpaste tablet for children. An example of the concentrations of different components is shown in the table of FIG. 1.

The children's toothpaste tablet employs a unique formulation designed especially for children's delicate oral care needs. The formula includes ingredients such as xylitol at about 15.00% by mass (w/w) of the composition, nano-hydroxyapatite of about 5.00% by mass (w/w) of the composition, and Rebaudioside A (Stevia) extract of about 0.15% by mass (w/w) of the composition, aimed at ensuring a gentle yet effective cleaning experience. Xylitol not only balances taste, but aids in maintaining a healthy oral pH, while nano-hydroxyapatite contributes to remineralizing teeth, ensuring their strength and health.

The toothpaste composition tablet for children may include glycerin in a concentration between about 22.5% and about 25.5% by mass (w/w) of the composition, and xylitol in a concentration between about 14.25% and about 15.75% by mass (w/w) of the composition. Sorbitol may be in a concentration between about 20.5% and about 23.5% by mass (w/w) of the composition, and nano-hydroxyapatite may be in a concentration between about 4.75% to about 5.25% by mass (w/w) of the composition. The toothpaste composition is formulated into a tablet form that may be less than 600 mg and closer to 250 mg to 350 mg.

Silicon dioxide may be in a concentration between about 9.0% and about 11.0% by mass (w/w) of the composition, and fused silica in a concentration between about 1.9% and about 2.1% by mass (w/w) of the composition as an added mixed ingredient to the composition. Xanthan gum may be in a concentration between about 0.38% and about 0.42% by mass (w/w) of the composition and cellulose gum in a concentration between about 3.8% and about 4.2% by mass (w/w) of the composition as an added mixed ingredient to the composition. A sweetening agent extract may be in a concentration between about 0.14% and about 0.16% by mass (w/w) of the composition. A flavoring agent may be in a concentration between about 2.85% and about 3.15% by mass (w/w) of the composition. The xylitol may include between about 30 and about 60 mesh particle size. Deionized water may be included.

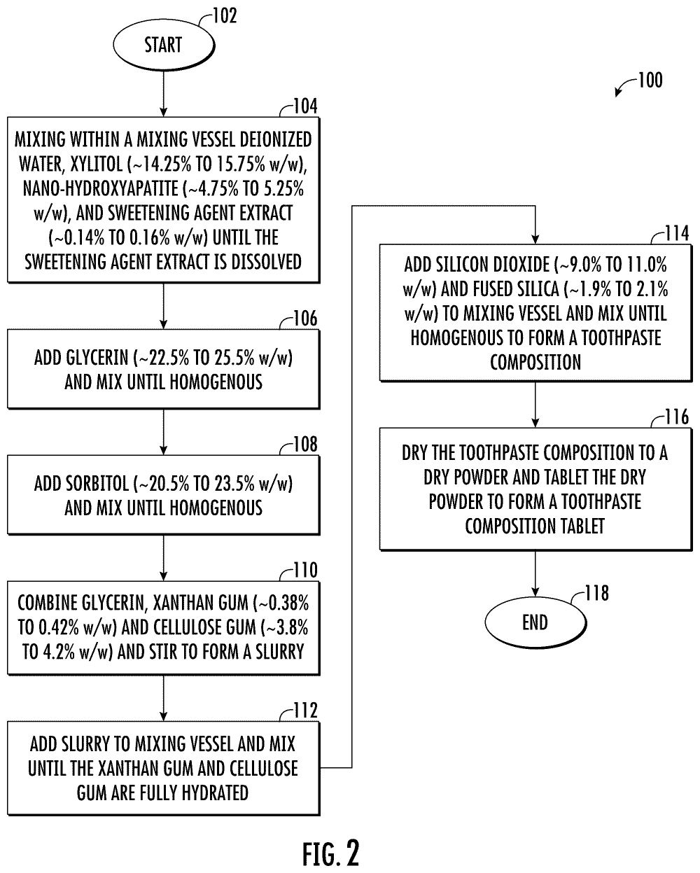

Referring now to FIG. 2, a high-level flowchart of the manufacturing process is shown generally at 100 and may generally be described with multiple phases to form the toothpaste composition tablet. The process starts (Block 102) and includes mixing within a mixing vessel deionized water, xylitol in a concentration between about 14.25% and about 15.75% by mass (w/w), nano-hydroxyapatite in a concentration between about 4.75% to about 5.25% by mass (w/w), and a sweetening agent extract in a concentration between about 0.14% and about 0.16% by mass (w/w) until the sweetening agent extract is dissolved (Block 104). This is followed by adding glycerin in a concentration between about 22.5% and about 25.5% by mass (w/w) to the mixing vessel and mixing until homogenous (Block 106), which is followed by adding sorbitol in a concentration between about 20.5% and about 23.5% by mass (w/w) to the mixing vessel and mixing until homogenous (Block 108).

This is followed by combining glycerin, xanthan gum in a concentration between about 0.38% and about 0.42% by mass (w/w) and cellulose gum in a concentration between about 3.8% and about 4.2% by mass (w/w) and stirring until smooth to form a slurry (Block 110). The slurry is added to the mixing vessel and mixed until the xanthan gum and cellulose gum are fully hydrated (Block 112). This is followed by adding silicon dioxide in a concentration between about 9.0% and about 11.0% by mass (w/w) and fused silica in a concentration between about 1.9% and about 2.1% by mass (w/w) to the mixing vessel and mixing until homogenous and smooth to form a toothpaste composition (Block 114). The process ends (Block 118).

The xylitol may be between about 30 and about 60 mesh particle size. A flavoring agent may be in a concentration between about 2.85% and about 3.15% by mass (w/w) and is added to the mixing vessel and mixed until homogenous before adding the silicon dioxide and fused silica. The sweetening agent extract may comprise Rebaudioside A.

The table shown in FIG. 1 gives a specific example of the ingredient concentrations that may be used. A systematic multi-phase mixing procedure as described above is used to ensure homogeneity and consistency as noted before. The process may begin with combining Phase A ingredients in a large mixing vessel, followed by the sequential addition of ingredients as per the specified phases (B, C, D, E, and F) with continuous mixing to ensure a smooth, homogeneous mixture. This structured approach reflects a keen attention to detail aimed at achieving a precise formulation with the desired texture and efficacy. For example, this structured approach in detail includes the following steps with the different phases as explained below:
A) In a large mixing vessel (vacuum mixing vessel preferred), combine phase A ingredients and mix until sweeteners have dissolved.
B) Add phase B to main tank while mixing. Mix until homogenous.
C) Add phase C to main tank while mixing. Mix until homogenous.
D) Combine phase D ingredients and stir until smooth. Add phase D slurry to main tank while mixing. Mix until gums are fully hydrated.
E) Add phase E ingredient to main tank while mixing. Mix until homogenous.
F) Add phase F ingredients to main tank while mixing. Mix until homogenous and smooth.

Glycerin, xanthan gum and cellulose gum are incorporated and mixed together to prevent the formation of “fish eyes” corresponding to “gooey” clumps, that form when gum hydrates unevenly. This helps in forming the homogenous mixture for later drying and tableting. The xanthan gum and cellulose gum may thicken the product and operate as a stabilizer. The glycerin also operates as a stabilizer that increases the flexibility during processing and may create space between polymer chains.

The glycerin helps disperse the xanthan and cellulose gums by creating a smooth slurry thanks to glycerin's humectant properties that helps water penetrate the gum structure. Also, glycerin can cross-link these gums to allow synergistic thickening effects. The glycerin also acts as a humectant to attract and retain moisture, which aids in hydration and dispersion of gums. The cellulose gum and xanthan gum are both hydrocolloids that operate as thickening agents to improve texture and appearance. The sorbitol may also operate as a humectant. Adding 10% silicon dioxide and 2% fumed silica improves flowability, prevents caking, and increases viscosity. The fumed silica has a higher surface area due to its smaller particle size, and thus, helps thicken during processing.

The children's toothpaste tablet's flavor profile is another notable feature. Unlike many children's toothpaste compositions that rely on artificial flavors, this toothpaste tablet is crafted with a natural bubblegum flavor of about 3.00% by mass (w/w) of the composition tablet, making brushing an enjoyable experience for children, while aligning with a commitment to natural, health-conscious ingredients.

Environmental considerations extend to the toothpaste tablet as well, with eco-friendly packaging, reflecting a broader commitment to ethical and sustainable practices. These considerations not only cater to the growing consumer demand for environmentally responsible products, but also sets the toothpaste tablet apart in a crowded market for children's oral care products.

The children's toothpaste tablet may have a range of components, with those components shown in the table of FIG. 1 and in percent mass (w/w) of the composition. The deionized water may range from about 15% to about 17% and the xylitol may be about 30/60 mesh during formation and range from about 14% to about 16%. Similar to the tablet described above with reference to Table I, the nano-hydroxyapatite may range from about 4.75% to about 5.25%. Because of the strength of the Rebaudioside A (stevia extract), the percent mass may range from a low of about 0.14% to 0.16%. These components are Phase A and mixed together.

The Phase B component as glycerin may range from about 22.5% to about 25.5%. Phase C as the sorbitol may range from about 21% to about 238. The glycerin used at Phase D may range in concentration similar to Phase B glycerin at about 22.5% to about 25.5%. The xanthan gum may range from about 0.39% to about 0.41%. The cellulose gum may range from about 3.5% to about 4.5%. The flavorant may range from about 2.5% to about 3.5% and the silicon dioxide may range from about 9.0% to about 11.0%. The fumed silicon may range from about 1.9% to about 2.1%.

Many of these components have been addressed above and it should be understood that the glycerin as a triol compound has antimicrobial and antiviral properties and is miscible with water and hygroscopic in nature. The sorbitol may also be referred to as glucitol and is a sugar alcohol with a sweet taste. The fumed silica is a strong thickening effect, an anti-caking agent, and may serve as a desiccant.

These ranges as described for different components are shown to vary by about 5% above and below the stated component percentages of the values shown in Table I above and the table of FIG. 1. The values may vary from about 6%, 7%, and in some instances 8%, 9% and 10% above and below these values.

A clinical trial was conducted with the toothpaste composition formulated into a tablet form as described above with reference to the composition such as shown in Table I, with the possible difference in ranges as described above for the different components, and more particularly, with the example including the nano-hydroxyapatite, calcium carbonate, anionic surfactant, such as from coconut oil, ammonium glycyrrhizinate, xylitol, sodium bicarbonate, and other ingredients. The example using glycerin, sorbitol and other ingredients as described with reference to the table in FIG. 1 is expected to also have similar beneficial results.

The clinical trial was a triple-blind, randomized controlled clinical study, to determine the efficacy of the toothpaste composition formulated into tablets, and hereafter “Toothpaste Composition Tablets” in improving symptoms of tooth sensitivity, breath freshness, and teeth cleanliness. A total of 70 participants were randomized into two groups. A first group received the Toothpaste Composition Tablets, and the second group received a placebo, each taken twice daily for four weeks. Participants completed the Dentine Hypersensitivity Experience Questionnaire (DHEQ-15) at Baseline, Week 1, Week 2, Week 3, and Week 4. This questionnaire is an established questionnaire known to those working in the study of dental hypersensitivity. In addition, perception questions about efficacy were asked at Day 1, Week 1, Week 2, Week 3 and Week 4.

The Toothpaste Composition Tablets are described above and contain about 5% nano-hydroxyapatite in combination with the other ingredients and their ranges to reduce tooth sensitivity and provide long-term protection to teeth. In addition, the beneficial balanced ingredient concentration includes the calcium carbonate, anionic surfactant from coconut oil, ammonium glycyrrhizinate, xylitol, and sodium bicarbonate and other ingredients described above to enhance tooth cleanliness in a safe, non-damaging way. Menthol and zinc citrate are also included in this example Toothpaste Composition Tablet to improve breath freshness, while the zinc citrate may form the complex with the nano-hydroxyapatite as explained above. The trial investigated whether the Toothpaste Composition Tablets, when used twice daily over four weeks, improved tooth sensitivity, breath freshness, and teeth cleanliness and other hygienic teeth improvement benefits.

Seventy (70) male or female participants aged 18 or over who experience tooth sensitivity were recruited for this study. All participants satisfied the following inclusion and exclusion criteria.

INCLUSION CRITERIA: Participants were male and female and 18 years old or over. The participants had difficulty with tooth sensitivity and pain or discomfort when consuming hot, cold, sweet, or acidic food and drinks, breathing through the mouth, or brushing teeth, and had concerns about bad breath.

The participants agreed to avoid introducing any products, prescription medications, or supplements that target tooth sensitivity, dental health, or oral hygiene for the duration of the study. They also agreed to retain their normal dental hygiene routine (i.e., flossing, mouthwash, and toothbrush) for the duration of the study. The participants agreed to drink a maximum of two cups of staining liquids (coffee, tea, or red wine) per day and agreed to refrain from increasing this for the duration of the study.

The participants had not used products, prescription medications, or supplements that target tooth sensitivity for at least one week prior to beginning the study.

EXCLUSION CRITERIA: The participants had no pre-existing chronic conditions that would prevent them from adhering to the protocol, including oncological and psychiatric disorders. The participants had no known severe allergic reactions that require an Epi-Pen or with allergies to any of the test or placebo product ingredients. The study excluded women who were pregnant, breastfeeding, or trying to conceive.

Participants had no dental work done in the past month before the study began or who had been planning to have any dental work done during the study period. None of the participants had braces (fixed or removable), dentures, crowns, or veneers. None of the participants had a history of recurrent oral thrush, oral cancer, or any other serious condition that affected the teeth, gums, or mouth, or had undergone an invasive medical procedure within the previous three months before the study began or had been planning to undergo an invasive medical procedure during the study period. None of the participants had a history of substance abuse. None of the participants smoked, vaped, or chewed tobacco or who had in the previous six months before the study began. None of the participants drank alcohol heavily (i.e., eight (8) or more alcoholic drinks per week for women, or 15 or more alcoholic drinks per week for men). All participants were willing to follow the study protocol.

This triple-blind trial required the participants to complete questionnaires at home. Before study onboarding, consent forms describing the study process, instructions, evaluation methods, and bill of rights were provided to the participants. Following the consent process, participants completed a baseline questionnaire, which included a validated DHEQ-15 questionnaire and study-specific questions about tooth sensitivity and general dental health/hygiene. Participants were randomized into one of two arms for the duration of the trial: 1) the Toothpaste Composition Tablet, or 2) a placebo product. Participants were not informed of the sponsor or intervention product name.

The validated questionnaire Dentine Hypersensitivity Experience Questionnaire (DHEQ-15) was used in this study. This was a clinical trial in which participants were required to use the Toothpaste Composition Tablets or the placebo product twice daily, in the morning and evening. Participants completed the questionnaires at baseline at the end of the first day of use, and at the end of Week 1, Week 2, Week 3, and Week 4. On Day 1, Week 1, Week 2, Week 3, and Week 4, the participants also completed participant perception questions determining their overall perception of the product's efficacy.

Data was collected from individual questions about tooth sensitivity and overall dental health/hygiene. Data was collected from the DHEQ-15 validated questionnaire and checked for normality using the Pearson test. To compare changes in participants' responses between the two groups, data for each participant at each time point was normalized to their respective baseline data and normalized data and reported as a percentage of baseline.

The results clarified the efficacy of the Toothpaste Composition Tablet relative to the placebo.

### On Day 1:

a) The Toothpaste Composition Tablet significantly improved overall dental health when compared with placebo;
b) 78.8% of participants agreed that the Toothpaste Composition Tablet was high quality;
c) 97% of participants agreed that the Toothpaste Composition Tablet was easy to use; and
d) 66.7% of participants agreed that since using the Toothpaste Composition Tablet, their breath had stayed fresh longer.

### At Week 1:

a) The Toothpaste Composition Tablet significantly improved the severity of tooth pain or discomfort experienced when compared with placebo;
b) 87.9% of participants agreed that the Toothpaste Composition Tablet was high quality;
c) 75.8% of participants agreed that since using the Toothpaste Composition Tablet, their teeth have felt cleaner;
d) 81.8% of participants agreed that since using the Toothpaste Composition Tablet, their breath had been fresher; and
e) 78.8% of participants agreed that since using the Toothpaste Composition Tablet, their breath had stayed fresh longer.

### At Week 2:

a) The Toothpaste Composition Tablet significantly improved the severity of tooth pain or discomfort experienced when compared with the placebo;
b) The Toothpaste Composition Tablet significantly improved their overall dental hygiene when compared with the placebo;
c) 84.8% of participants agreed that the Toothpaste Composition Tablet was high quality;
d) 84.8% of participants agreed that since using the Toothpaste Composition Tablet, their teeth had felt cleaner; and
e) 87.9% of participants agreed that since using the Toothpaste Composition Tablet, their breath had stayed fresh longer.

### At Week 3:

a) 93.9% of participants agreed that the Toothpaste Composition Tablet was high quality;
b) 81.8% of participants agreed that since using the Toothpaste Composition Tablet, their teeth had felt cleaner;
c) 87.9% of participants agreed that since using the Toothpaste Composition Tablet, their breath had been fresher;
d) 84.8% of participants agreed that since using the Toothpaste Composition Tablet, they had experienced reduced symptoms of tooth sensitivity;
e) 78.8% of participants agreed that since using the Toothpaste Composition Tablet, they felt that their oral health had improved; and
f) 84.8% of participants agreed that since using the Toothpaste Composition Tablet, they felt that their dental hygiene had improved.

### At Week 4:

a) The Toothpaste Composition Tablet significantly improved the severity of tooth pain or discomfort experienced when compared with the placebo;
b) 90.9% of participants agreed that the Toothpaste Composition Tablet was high quality;
c) 78.8% of participants agreed that since using the Toothpaste Composition Tablet, their teeth have felt cleaner;
d) 87.9% of participants agreed that since using the Toothpaste Composition Tablet, their breath had been fresher;
e) 84.8% of participants agreed that since using the Toothpaste Composition Tablet, their breath had stayed fresh longer;
f) 84.8% of participants agreed that since using the Toothpaste Composition Tablet, they had experienced reduced symptoms of tooth sensitivity;
g) 84.8% of participants agreed that since using the Toothpaste Composition Tablet, they felt that their oral health had improved;
h) 87.9% of participants agreed that since using the Toothpaste Composition Tablet, they felt that their dental hygiene had improved;
i) 90.9% of participants agreed that they were satisfied with the results of the Toothpaste Composition Tablet;
j) 84.8% of participants agreed that they would like to continue using the Toothpaste Composition Tablet;
k) 87.9% of participants agreed that they would purchase the Toothpaste Composition Tablet; and
1) 87.9% of participants agreed that they would recommend the Toothpaste Composition Tablet to friends and family.

The clinical trial successfully validates the effectiveness of the Toothpaste Composition Tablet.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims.

1. A toothpaste composition formulated as a tablet, comprising:
xylitol in a concentration between about 48.5% and about 53.5% by mass (w/w) of the composition;
a flavoring agent in a concentration between about 11.4% and about 12.6% by mass (w/w) of the composition;
cellulose in a concentration between about 11.2% and about 12.3% by mass (w/w) of the composition;
calcium carbonate in a concentration between about 6.2% and about 6.8% by mass (w/w) of the composition;
an anionic surfactant in a concentration between about 5.0% and about 5.6% by mass (w/w) of the composition; and
nano-hydroxyapatite in a concentration between about 4.75% and about 5.25% by mass (w/w) of the composition; and
wherein the toothpaste composition is formulated into a tablet form.

2. The toothpaste composition of claim 1 comprising xanthan gum in a concentration between about 1.9% and about 2.1% by mass (w/w) of the composition.

3. The toothpaste composition of claim 1 comprising sodium cocoyl isethionate (SCI) in a concentration between about 1.8% and about 2.0% by mass (w/w) of the composition.

4. The toothpaste composition of claim 1 comprising ammonium glycyrrhizinate in a concentration between about 1.8% and about 2.0%. by mass (w/w) of the composition.

5. The toothpaste composition of claim 1 comprising silica dioxide in a concentration between about 1.05% and about 1.2% by mass (w/w) of the composition.

6. The toothpaste composition of claim 1 comprising at least one of the following: menthol, zinc citrate and sodium bicarbonate.

7. A toothpaste composition formulated as a tablet, comprising:
glycerin in a concentration between about 22.5% and about 25.5% by mass (w/w) of the composition;
xylitol in a concentration between about 14.25% and about 15.75% by mass (w/w) of the composition;
sorbitol in a concentration between about 20.5% and about 23.5% by mass (w/w) of the composition; and
nano-hydroxyapatite in a concentration between about 4.75% to about 5.25% by mass (w/w) of the composition; and
wherein the toothpaste composition is formulated into a tablet form.

8. The toothpaste composition of claim 7 comprising silicon dioxide in a concentration between about 9.0% and about 11.0% by mass (w/w) of the composition and fused silica in a concentration between about 1.9% and about 2.1% by mass (w/w) of the composition as an added mixed ingredient to the composition.

9. The toothpaste composition of claim 7 comprising xanthan gum in a concentration between about 0.38% and about 0.42% by mass (w/w) of the composition and cellulose gum in a concentration between about 3.8% and about 4.2% by mass (w/w) of the composition as an added mixed ingredient to the composition.

10. The toothpaste composition of claim 7 comprising a sweetening agent extract in a concentration between about 0.14% and about 0.16% by mass (w/w) of the composition.

11. The toothpaste composition of claim 7 comprising a flavoring agent in a concentration between about 2.85% and about 3.15% by mass (w/w) of the composition.

12. The toothpaste composition of claim 7 wherein the xylitol comprises between about 30 and about 60 mesh particle size.

13. The toothpaste composition of claim 7 comprising deionized water.

14. A method of forming a toothpaste composition as a toothpaste tablet, comprising:
mixing within a mixing vessel deionized water, xylitol in a concentration between about 14.25% and about 15.75% by mass (w/w), nano-hydroxyapatite in a concentration between about 4.75% to about 5.25% by mass (w/w), and a sweetening agent extract in a concentration between about 0.14% and about 0.16% by mass (w/w) until the sweetening agent extract is dissolved, followed by;
adding glycerin in a concentration between about 22.5% and about 25.5% by mass (w/w) to the mixing vessel and mixing until homogenous, followed by;
adding sorbitol in a concentration between about 20.5% and about 23.5% by mass (w/w) to the mixing vessel and mixing until homogenous, followed by;
combining glycerin, xanthan gum in a concentration between about 0.38% and about 0.42% by mass (w/w) and cellulose gum in a concentration between about 3.8% and about 4.2% by mass (w/w) and stirring until smooth to form a slurry;
adding the slurry to the mixing vessel and mixing until the xanthan gum and cellulose gum are fully hydrated, followed by;
adding silicon dioxide in a concentration between about 9.0% and about 11.0% by mass (w/w) and fused silica in a concentration between about 1.9% and about 2.1% by mass (w/w) to the mixing vessel and mixing until homogenous and smooth to form a toothpaste composition; and
tableting the toothpaste composition.

15. The method of claim 14 wherein the xylitol comprises between about 30 and about 60 mesh particle size.

16. The method of claim 14 wherein a flavoring agent in a concentration between about 2.85% and about 3.15% by mass (w/w) is added to the mixing vessel and mixed until homogenous before adding the silicon dioxide and fused silica.

17. The method of claim 14 wherein the sweetening agent extract comprises Rebaudioside A.

18. The method of claim 14 comprising drying the toothpaste composition to a dry powder and tableting the dry powder to form the toothpaste composition tablet.
